(19) United States
(12) Patent Application Publication
(10) Pub. No.: US 2003/0027176 A1
(43) Pub. Date: Feb. 6, 2003

(54) Innate immunity markers for rapid diagnosis of infectious diseases

(76) Inventor: Peter J. Dailey, Clayton, CA (US)

Correspondence Address: REED & ASSOCIATES, 800 MENLO AVENUE, SUITE 210, MENLO PARK, CA, 94025, US

(21) Appl. No.: 10/077,680

(22) Filed: Feb. 15, 2002

Related U.S. Application Data
(60) Provisional application No. 60/269,294, filed on Feb. 15, 2001.

(51) Int. Cl. C12Q 1/68

(52) U.S. Cl. 435/6

(57) ABSTRACT

A method is provided for determining the type of an infectious pathogen in a patient who is suspected to be suffering from an infectious pathogen. The method involves first measuring the amounts of a plurality of markers in a body fluid sample of the patient. The markers of interest are produced by the patient as part of that patient's innate immune response to the presence of the infectious pathogen and are indicative of the type of the infectious pathogen in the patient. Next, a marker profile is identified based on the measured amounts of the plurality of markers. Finally, if the marker profile is indicative of an infection, then the type of infectious pathogen within the patient is determined from the marker profile. In preferred embodiments, any individual marker is either an mRNA or a protein. Methods for identifying suitable markers and kits are provided as well.

### CROSS-REFERENCE TO RELATED APPLICATION

This application claims priority under 35 U.S.C. §119(e)(1) to U.S. Provisional Application Serial No. 60/269,294, filed Feb. 15, 2001.

### TECHNICAL FIELD

The present invention relates generally to the diagnosis of disease, and more specifically relates to novel methods for identifying and using markers associated with an individual's innate immunity system, wherein the markers serve as a basis to determine the presence and/or to identity the type of an infectious pathogen in a patient. The invention has utility in the fields of diagnostics, diagnostic assays and medicine.

### BACKGROUND

Health care professionals require accurate and expedient methods for diagnosing ill patients under their care. Such methods allow the health care professional to provide aggressive and appropriate medical treatment, particularly for critically ill patients.

Often, medical diagnoses are carried out by a health care professional drawing upon his or her own clinical experience and knowledge and forming a conclusion on the likely etiology of a patient's disease state. Although expedient, a health care professional's diagnosis based on the observation of only a few symptoms may be erroneous, particularly when different disease states present with similar or identical clinical indicators. Furthermore, the health care professional is unlikely to be able to correctly diagnose a disease state that he or she has never previously treated. Consequently, health care professionals often substitute or supplement their own preliminary conclusions concerning a patient's disease state by relying on the results of one or more diagnostic assays designed to detect or identify the cause of a patient's illness.

Although early assays were relatively simple, e.g., measuring the temperature of a patient with a thermometer, recent advances in science and technology have greatly expanded the sophistication and number of diagnostic assays available to the health care professional. Currently, laboratory technicians can determine both the amounts and types of white blood cells present in a patient's peripheral circulation by using microscopy to view a blood sample. The number of white blood cells and differentiation of white blood cells per unit volume is useful in establishing the presence of a microbial infection in a patient. Samples of body fluid, e.g., sputum, urine, blood and wound samples, can be cultured on suitable plates, e.g., agar plates, so that bacteria, if present in the sample, can be detected and identified. In addition, certain viral infections (such as hepatitis C (HCV)) can be identified using assays such as the Versant™ HCV RNA Qualitative Assay (Bayer Diagnostics, Tarrytown, N.Y.). Clearly, these assays and procedures assist the clinician in correctly diagnosing diseases, which, in turn, can ensure more appropriate treatment.

Many conventional diagnostic assays and procedures used to identify the presence of infections, however, are nonspecific, slow or inaccurate. For example, assays that measure C-reactive protein are often used as an indicator for appendicitis, pneumonia and other illnesses. Such nonspecific assays, however, are not useful in critical-care situations where immediate treatment is required. Clyne et al. (1999) J. Emerg. Med. 17(6):1019-1025. In addition, assays that measure the erythrocyte sedimentation rate may indicate changes in protein content of blood and blood cells, but the cause, e.g., infection, arthritis, etc., cannot be determined without further testing. Thus, such nonspecific assays and procedures are unable to provide the clinician with an unequivocal determination concerning the presence of an infection.

As stated above, diagnostic assays used to determine infectious diseases are often slow. Assays that rely on culturing the organism, for example, are slow as the outcome of the assay is delayed until the culture grows to a detectable level. In addition, assays that detect moieties developed by the patient's adaptive immune system in response to the presence of the infectious pathogen are also slow. Exemplary of this type are assays that detect the presence of specific antibodies, e.g., antibodies to hepatitis C virus (HCV), which necessarily rely on the patient's own immune system to develop those antibodies. The delay associated with the development of antibodies in a patient may cause a false negative in an assay that detects antibodies, which, in turn, may cause the clinician to refrain from initiating therapy.

In addition, many assays that detect the presence of the infectious pathogen are often inaccurate. For example, those assays that directly detect the presence of bacteria in a sample may result in false negatives when the bacteria are present in amounts below the detection threshold of the assay. Similarly, false negative results may occur when patients receive subtherapeutic therapy, e.g., receiving a subtherapeutic dose of an antibacterial agent, as the amount of the infectious pathogen is reduced to below detectable levels.

Particularly for very ill patients, a nonspecific, delayed or inaccurate diagnosis may result in delayed and/or inappropriate treatment that can lead to further complications or even death. Inappropriate antibiotic administration, for example, may also result in development of antibiotic-resistant strains of bacteria. Furthermore, a delayed diagnosis has been found to increase the overall cost for treating infected patients. Barenfanger et al. (2000) J. Clin. Microbiol. 38(8):2824-2828.

Both direct detection of the infectious pathogen and indirect detection of moieties produced by the patient as part of the adaptive immune system are ineffective during the early stages of infection. It is in these early stages of infection, however, that patients would most benefit from a rapid and accurate diagnosis. Such an expedient diagnosis would allow for aggressive and appropriate treatment to eradicate the pathogen, decrease symptoms, and/or reduce further complications.

As its name suggests, innate immunity is possessed at birth. Innate immunity is comprised of several mechanisms designed to defend and fight against infectious pathogens. One of the many aspects of the innate response involves the rapid, direct recognition of pathogen-associated molecular patterns (PAMPs) present on pathogens or in infected cells. These PAMPs are consensus molecular structures of pathogens that essentially provide a “molecular footprint” identifying the type of infectious pathogen, e.g., gram-positive bacteria, gram-negative bacteria, virus, fungus, etc. Cells associated with the innate immune response have receptors that recognize these PAMPs. Some of these receptors have been designated Toll-like receptors and are believed to recognize specific PAMPs. International publications WO 98/50547 and WO 99/20756 describe several Toll-like receptors. Once activated by a particular PAMP, the appropriate receptor triggers a cascade of events and the production of certain moieties that lead to the production of specific proteins designed to assist in the patient's fight against the infectious pathogen. This particular response by the innate immune system is immediate and may be complete within minutes to several hours of after exposure to the infectious pathogen.

Thus, assays that are designed to measure a plurality of markers or signals corresponding to the innate immune response should be specific for a particular infectious pathogen and allow for an early diagnosis. EP 0725081 describes using human Mx protein MxA monoclonal antibodies in the diagnosis of viral infections. It has been found, however, that basing a diagnosis on a single infectious indicator is insufficient and that two or more indicators are required to provide an accurate diagnosis of infection. In contrast, previous disclosures such as that provided in EP 0725081 do not describe diagnostic procedures or assays relying on a plurality of signals or markers of the innate immune system. The development of such assays therefore represents an important advance in the field of diagnostic assays and medicine. The present invention satisfies this and other needs in the art.

### SUMMARY OF THE INVENTION

Accordingly, it is a primary object of the invention to address the above-described need in the art by providing a method for determining the type of an infectious pathogen in a patient by measuring the quantity of each of a plurality of markers in a specimen obtained from a patient, identifying a marker profile therefrom, and determining the type of infectious pathogen, if present, based on the marker profile.

It is another object of the invention to provide such a method wherein the plurality of markers are selected from the group consisting of a messenger ribonucleic acid (mRNA), a protein, and combinations thereof, wherein each marker is produced as a result of the patient's innate immune system in response to the presence of the invading pathogen.

It is yet another object of the invention to provide an assay kit for determining the presence of an infectious pathogen in a patient.

It is still another object of the invention to provide a method for identifying the markers that are indicative of the presence of an infectious pathogen in a patient

Additional objects, advantages and novel features of the invention will be set forth in part in the description which follows, and in part will become apparent to those skilled in the art upon examination of the following, or may be learned by practice of the invention.

In one aspect of the invention then, a method is provided for determining the type or identity of an infectious pathogen in a patient who is suspected to be suffering from an infection. The method involves determining the amount of each of a plurality of markers in a specimen obtained from the patient. Each marker, typically an mRNA or a protein, is produced by the patient as part of the innate immune response to the presence of the infectious pathogen. Once each marker is quantified, a marker profile is identified based on the measured amount of each of the plurality of markers. The marker profile is determined using a priori quantitative or qualitative designations for each marker. For qualitative designations, the marker is compared against previously established controls and assigned a certain designation, e.g., normal or abnormal. For quantitative designations, each marker measured is designated with a numerical value. Each individual marker included in the marker profile may be assigned only a quantitative designation, only a qualitative designation, or a combination of both. Finally, if the marker profile is indicative of an infection, then the type of infectious pathogen is determined from the marker profile. This step is generally performed by comparing the marker profile obtained from the patient specimen to known profiles or patterns associated with a certain type of infectious pathogen. Generally, although not necessarily, profiles or patterns of markers associated with a certain type of pathogen are obtained from measuring the same markers obtained from a patient known to have a certain type of infection.

In a related aspect of the invention, an assay and assay kit are provided for determining the presence of an infectious pathogen in a patient. The assay kit includes (a) a plurality of biomolecular probes, e.g., oligonucleotide probes or antibodies, (b) a plurality of label probes, and (c) written instructions for carrying out the assay. Each biomolecular probe is complementary to a first region of a different marker that is at least partially (e.g., no necessarily conclusively) indicative of the presence of the infectious pathogen. Under binding conditions (e.g., hybridizing conditions for oligonucleotides, antibody binding conditions for immunoassays, etc.), each biomolecular probe forms a probe-marker complex by binding to the first region of a marker specific for that particular probe. Each label biomolecular probe has a region that binds to either a region on a probe-marker complex or a region of an intermediary biomolecular probe that is directly or indirectly coupled to a probe-marker complex. Thus, if the probe is labeled, the complex can be detected directly. When the probe is not labeled, additional layers of probe can provide for indirect detection of the marker. In either case, the presence of the label probe provides the ability to detect and measure the presence of a particular marker in the sample.

As will be discussed in further detail below, detecting and measuring particular markers may be accomplished using any art-known procedure and provided in any number of assay formats. When the marker is an mRNA, preferred assays and techniques include a sandwich hybridization, branched-oligonucleotide hybridization, Northern blot, a solution phase assay (e.g., fluorescent resonance energy transfer assay “FRET assay”), reverse transcriptase-polymerase chain reaction (RT-PCR), transcription-mediated amplification (TMA), nucleic acid sequence-based amplification or (NASBA®) and RNAse protection assay. Alternatively, when the marker is a protein, immunoassay, centrifugation, electrophoresis, enzyme immunoassay, high performance liquid chromatography (HPLC), size exclusion chromatography, solid-phase affinity and Western blotting are preferably used.

In yet another aspect of the invention, a method is provided for identifying a marker that is at least partially indicative of the presence of an infectious pathogen in a patient. Initially, the method comprises comparing (a) the expression of genes in a patient specimen, e.g., a sample containing a white blood cell, taken from a patient who is infected with the infectious pathogen to (b) the expression of genes of a specimen taken from an individual who is not infected. By comparing the two, i.e., determining which genes are expressed in the specimen taken from an infected patient and comparing the results to that of the uninfected individual, it is possible to identify those genes of the innate immune system that become expressed upon exposure to a particular infectious pathogen. From such information, it is possible to determine those markers, e.g., mRNAs or proteins, that are suitable for use as “identifiers” for a particular type of infectious pathogen. The procedure may be repeated with specimens taken from patients suffering from other types of infectious pathogens, e.g., microbes, fungal organisms and viruses, to determine additional markers.

In still another aspect of the invention, an additional method for identifying markers is provided. The method is used to identify protein markers by comparing (a) the proteins present in a patient specimen, e.g., a sample of body fluid, taken from a patient who is infected with the infectious pathogen to (b) the proteins present in a specimen taken from an individual who is not infected with the infectious pathogen. A protein that is present in (a) and not in (b) represents a protein marker that is indicative of the presence of an infectious pathogen. Preferably, comparison of proteins is carried out using gel electrophoresis.

### BRIEF DESCRIPTION OF THE DRAWINGS

FIG. 1A is a graph depicting the amounts of two mRNA markers in blood samples from an individual with no infection, an individual with a viral infection, and an individual with an infection of gram-positive (+) bacteria.

FIG. 1B is a graph depicting the amounts of two mRNA markers in a blood sample from a patient suspected to be suffering from an infectious pathogen.

### DESCRIPTION OF THE INVENTION

Definitions and Overview:

Before describing the present invention in detail, it is to be understood that unless otherwise indicated this invention is not limited to specific markers, assays, pathogens, or the like, as such may vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “a probe” includes a single probe and two or more identical or different probes, reference to a “marker” refers to a single marker or two or more identical or different markers, and the like.

In this specification and in the claims that follow, the following terminology will be used in accordance with the definitions set forth below.

A “marker” is a moiety produced by a cell in response to exposure to a particular type infectious pathogen. The marker is associated with the innate immune response of the individual. As will be appreciated, a vast number of markers are produced during the innate immune response. The markers used in the present invention are those that, in combination with other markers, are used to determine a type of infectious pathogen. Thus, a plurality of markers forming a marker profile is used to determine the type of infectious pathogen according to the present method. Typically, although not necessarily, the markers are mRNAs and/or proteins.

“Patient” as used herein refers to an organism, preferably mammalian, more preferably human, possessing innate immunity. The present invention provides for determining the type of infectious pathogen present in an infected patient.

As used herein, the terms “patient specimen,” “a specimen obtained from a patient” and “a specimen obtained from an individual” are used interchangeably and include any sample obtained from a patient or other individual possessing innate immunity. Thus, the specimen may be a solid tissue sample, e.g., a sample of tissue obtained from a biopsy, a fluid sample, e.g., a blood sample, or any other patient specimen commonly used in the medical community. In some embodiments of the invention, the specimen, including specimens of “body fluid,” is a sample of lymph fluid, lysates of cells, milk, plasma, saliva, semen, serum, spinal fluid, tears, whole blood, fractions of whole blood, wound samples, the external sections of the skin, and the secretions of the respiratory, intestinal, and genitourinary tracts. Preferably, the specimen is blood, sputum, urine or fractions of whole blood.

“Oligonucleotide” shall be generic to polydeoxyribonucleotides (containing 2′-deoxy-D-ribose or modified forms thereof), to polyribonucleotides (containing D-ribose or modified forms thereof), and to any other type of polynucleotide which is an N-glycoside of a purine or pyrimidine base, or of a modified purine or pyrimidine base. The oligonucleotides may be single-stranded or double-stranded, typically single-stranded. Also, the oligonucleotides used in the present invention are normally of from about 2 to about 2000 monomer units, more typically from about 2 to about 100 monomer units, and most typically from about 2 to about 60 monomer units.

As used herein, the term “biomolecular probe” refers to a structure that can bind to a marker, either directly or indirectly. The biomolecular probe is preferably an oligonucleotide or antibody. Oligonucleotides that function as biomolecular probes have a structure comprised of an oligonucleotide, as defined above, which contains a nucleic acid sequence complementary to a region of a target nucleotide sequence (e.g., a marker), at least one probe, or both. The oligonucleotide regions of the probes may be composed of DNA, and/or RNA, and/or synthetic nucleotide analogs. Antibodies, fragments of antibodies and phage display of antibodies also function as “biomolecular probes” and may be an immunoglobulin such as IgG, IgD, IgA, IgE or IgM that can bind to molecule, e.g., a protein, that serves as a marker. Thus, for use herein, the term “antibodies” includes whole antibodies, fragments of antibodies and phage display of antibodies. Included within biomolecular probes are “label probes,” and “intermediary biomolecular probes.”

It will be appreciated that the binding sequences of oligonucleotide probes need not have perfect complementarity to provide stable hybrids. In many situations, stable hybrids will form where fewer than about 10% of the bases are mismatches, ignoring loops of four or more nucleotides. Accordingly, the term “substantially complementary” refers to an oligonucleotide that forms a stable duplex with its “complement” under assay conditions, generally where there is about 90% or greater homology.

The term “binding conditions” is intended to mean those conditions of time, temperature and pH and the necessary amounts and concentrations of reactants and reagents sufficient to allow binding between binding pairs, e.g., an oligonucleotide to hybridize with an oligonucleotide having a complementary sequence or an antibody to a protein having the corresponding epitope. As is well known in the art, the time, temperature and pH conditions required to accomplish binding depend on the size of each member of the binding pair, the affinity between the binding pair, and the presence of other materials in the reaction admixture. The actual conditions necessary for each binding step are well known in the art or can be determined without undue experimentation.

Typical binding conditions for most biomolecules, e.g., complementary oligonucleotides and antibodies to a protein having the necessary epitope, include the use of solutions buffered to a pH from about 7 to about 8.5, and are carried out at temperatures from about 22° C. to about 60° C. and preferably from about 30° C. to about 55° C. for a time period of from about 1 second to about 1 day, preferably from about 10 minutes to about 16 hours, and most preferably from about 15 minutes to about 3 hours.

“Binding conditions” also require an effective buffer. Any buffer that is compatible, i.e., chemically inert, with respect to biomolecules and other components, yet still allows for binding between the binding pair, can be used.

Unless the context clearly indicates otherwise, the term “protein” intends a polymer in which the monomers are amino acids linked together through amide bonds. The protein may be composed of at least about 5 amino acids, more usually at least about 10 amino acids, and most usually at least about 50 amino acids.

“Optional” or “optionally” means that the subsequently described circumstance may or may not occur, so that the description includes instances where the circumstance does occur and instances where it does not.

The term “coupled” as used herein refers to attachment by covalent bonds or by non-covalent interactions (e.g., hydrophobic interactions, hydrogen bonds, etc.). Covalent bonds may be, for example, ester, ether, phosphoester, amide, peptide, imide, carbon-sulfur bonds, carbon-phosphorus bonds, and the like. Methods for coupling oligonucleotides and proteins to substrates are known in the art and include, for example, blotting of the oligonucleotide or protein onto the substrate.

The term “substrate” refers to any solid or semi-solid surface to which a desired binding partner may be anchored. Suitable substrate materials may be any material that can immobilize a biomolecule, e.g., an oligonucleotide or protein, and includes, for example, glass (e.g., for slides), nitrocellulose (e.g., in membranes), plastics including polyvinyl chloride (e.g., in sheets or microtiter wells), polystyrene latex (e.g., in beads or microtiter plates), polyvinylidine fluoride (e.g., in microtiter plates), and polystyrene (e.g., in beads), metal, polymer gels, and the like.

The term “label” as used herein refers to any atom or moiety that can be used to provide a detectable (preferably quantifiable) signal, and that can be attached to a biomolecule, e.g., an oligonucleotide or protein.

As used herein, the terms “label biomolecular probe” and “label probe” refer to a biomolecular probe in which the biomolecule is coupled to a label either directly, or indirectly via a set of ligand molecules with specificity for each other.

By “type of infectious pathogen,” as in “determining the type of infectious pathogen,” is intended the identification of a class of infectious pathogens or species of a particular infectious pathogen. Thus, for example, determining a “type of infectious pathogen” includes determining whether a patient is suffering from a particular class of infection, e.g., a bacterial, yeast, viral or fungal infection. Such a class may be any commonly used class that organizes various types of infectious organisms or may be a specific taxonomic class, e.g., family, genus, etc. More specific identifications such as determining gram-positive bacterial or gram-negative bacterial infections are also contemplated. Furthermore, determination of the “type of infectious pathogen” also includes identification of the actual species of the infectious pathogen, e.g., Staphylococcus aureus, Heamophilus influenzae, Listeria monocytogenes, Salmonella Dublin, Escherichia coli, Bordetella pertussis, and the like.

Determination of the Cause of Infection in a Patient:

In a first embodiment, the invention provides a method for determining the type of an infectious pathogen in a patient who is suspected to be suffering from an infection. The method generally employs known techniques to detect and quantitated each of a plurality of markers, e.g., mRNA markers, protein markers or a combination thereof. The particular mRNAs or proteins (or other biomolecules) that are measured are markers that indicate the type of infectious pathogen causing the patient's illness. According to the present invention, a plurality of markers, e.g., two, three, four, or more markers, is used in determining the type of infectious pathogen in a patient. Although there is no limit to the number of markers used, it is preferred that no more than about 12 markers be used to make a diagnosis, i.e., to confirm or rule out any given type of infection. As illustrated below in Example 1, measurement of a single biological parameter is insufficient to determine the type of infectious pathogen in a patient.

The method comprises measuring the amounts of a plurality of markers in a specimen obtained from the patient, wherein each of the markers of interest is produced by the patient and represents a response by the patient's innate immune system to the presence of the infectious pathogen. In addition, each of the markers must at least partially be indicative of the type of infectious pathogen in the patient. Once the appropriate markers have been measured, a marker profile is identified based on the amounts of each of the plurality of markers. The profile may be based on a quantitative designation for each marker, a qualitative designation for each marker, or combination of both. Thereafter, if the marker profile obtained from the sample is indicative of an infection, a further step involves the determination of the type of infectious pathogen. In order to make this determination, the marker profile is compared to a library of known profiles previously documented as indicative of a particular type of infection. A substantial or exact match between the two, i.e., the marker profile obtained from an individual suspected of suffering from an infection and one documented to identify a type of infection, indicates that the individual is suffering from that type of infection. For example, a substantial match between an individual's marker profile and a profile designating a gram-negative bacterial infection indicates that the individual is suffering from a gram-negative bacterial infection.

The invention relates to the discovery that the innate immune system, traditionally thought of as being nonspecific, can discriminate between different types of infectious pathogens. Among other things, a patient's innate immune system discriminates between various types of infectious pathogens by using “pattern-recognition receptors” that are expressed on effector cells, e.g., monocytes, macrophages, dendritic cells and natural killer (NK) cells. Present in most if not all multicellular organisms, pattern-recognition receptors are encoded in the germ line of multicellular organisms, thereby providing the “innate” quality of this pathogenic defense system. In the fruit fly, Drosophila melanogester, the pattern-recognition receptors include “Toll receptors,” while in mammalian organisms, including humans, the corresponding receptors have been designated “Toll-like receptors” or “TLRs” due to similar structure and function. Kopp et al. (1999) Curr. Opin. Immunol. 11(1):13-18.

Toll receptors and TLRs recognize specific “pathogen-associated molecular patterns” or “PAMPs” that are present on the infectious pathogen itself. Each PAMP is unique to a particular infectious pathogen or class of infection pathogens. Exemplary PAMPs include lipoteichoic acid (gram-positive bacilli), lipopolysaccharide (gram-negative bacteria), peptidoglycan (gram-positive and gram-negative bacilli), mannans (yeast), muramyl peptide (mycobacteria), and double-stranded RNA (viruses).

Once a pattern-recognition receptor binds to a complementary PAMP, effector cells, e.g., white blood cells, immediately initiate an immune response appropriate for that type of infectious pathogen, such as, for example, up-regulating proteins having antimicrobial activity when a pattern-recognition receptor binds to microbial PAMP. As mRNA is required for the production of proteins involved in such an immune response, mRNA and/or the expressed protein are used as one of a plurality of “markers” for determining the type of infectious pathogen causing illness in a patient. Thus, measuring an mRNA encoding an antibacterial protein or an antibacterial protein associated with the innate immune response along with other appropriate markers will indicate that the infectious pathogen is bacterial in nature, and not, for example, fungal or viral. Other groups of markers serve as indicators for other types of infectious pathogens. Markers other than a mRNA or a protein may be used, however, mRNA markers and protein markers are preferred.

mRNA Markers:

Before measuring mRNA, a patient specimen is obtained. Preferably, although not necessarily, the patient specimen is a sample of body fluid is taken from a patient. Although any body fluid may be used, it is preferred that the body fluid contains white blood cells. It is also preferred that the body fluid is blood, sputum or urine. Any art-known methods for obtaining the patient specimen may be used. Procedures for obtaining blood samples, for example, include withdrawing venous blood with a conventional syringe and needle. Sputum samples may also be obtained using any art-known method including brochoalveolar lavage. Often, the sample will contain white blood cells such as monocytes, dendritic cells, lymphocytes, polymorphonuclear leukocytes and combinations thereof, which are preferred for use in accordance with the present method. When white blood cells are present in the patient specimen, it is preferred that the specimen contains from about 10,000 to about 10,000,000 white blood cells. It is expected that about 10,000,000 white blood cells will contain about 1-5 μg of mRNA, which is sufficient for the presently described methods.

The patient specimen is generally treated with reagents to preserve mRNA and/or to assist in the carrying out the assay. In particular, it is preferred to add a ribonuclease inhibitor (RNase inhibitor) to decrease the digestion of mRNA by RNases present in the sample, particularly in the cytosol of white blood cells, for example. RNase inhibitors are well-known in the art and are commercially available. Examples of RNase inhibitors include, but are not limited to, Prime RNase inhibitor (available from Eppendorf Scientific, Inc., Westbury, N.Y.), human placental RNase inhibitor and ribonuclease vanadyl complexes (both available from Sigma Corp., St. Louis, Mo.), Superase In RNase inhibitor (Ambion Corp., Austin Tex.), and RNasin® RNase inhibitor (Promega Corp., Madison, Wis.). In addition, RNase-free DNase (Promega Corp., Madison, Wis.) may optionally be added to digest DNA and thereby reduce the potential interference of DNA during mRNA measurement.

When present, white blood cells and other cells contained within the patient specimen may be lysed, although lysing is not required. During the optional lysing step, care must be taken so the sample is not subjected to conditions harsh enough to destroy mRNA. Such methods are also well-known in the art. Examples of preferred lytic techniques include, but are not limited to, subjecting the sample to a lysis buffer (e.g., a buffer containing Proteinase K or a guanidine isothiocyanate buffer, both available from Sigma Corp., St. Louis, Mo.).

The lysate may then be treated such that nonRNA matter is discarded so that the sample contains substantially only RNA. Such treatments are well-known in the art. For example, the RNA in the lysate may be collected by sequential ethanol precipitation. Chirgwin et al. (1979) Biochemistry 18:5290-5294. The mRNA that is present in the sample is retained, while the remainder, e.g., organelles originally contained in the cells, is discarded. Once the sample is prepared, mRNA contained in the sample is available to participate in oligonucleotide hybridization.

Whole cells may also be used according the present methods and analyzed through flow cytometry techniques, thereby decreasing assay preparation time. In this way, the time period between obtaining the patient specimen and providing the diagnosis is reduced.

Although mRNA may be measured by any number of procedures, the present invention provides for mRNA quantitation using a nucleic acid assay. As is known in the art, nucleic acid assays are based on oligonucleotide hybridization techniques. Any type of art-known nucleic acid assay that can be adopted to measure mRNA in a patient sample may be used. Such assays include, for example, sandwich hybridization, branched-oligonucleotide hybridization, Northern blot, solution phase assay (e.g., fluorescent resonance energy transfer assay or “FRET” assay), reverse transcriptase-polymerase chain reaction, transcription-mediated amplification, nucleic acid sequence-based amplication or and RNAse protection assay.

A variety of sandwich hybridization assays are known. See, for example, U.S. Pat. Nos. 5,124,246, 5,710,264 and 5,849,481 to Urdea et al. Briefly, the mRNA-containing patient specimen is placed in contact with oligonucleotide probes under hybridizing conditions. The oligonucleotide probes then hybridize to a first region of the mRNA to form an oligonucleotide probe-mRNA complex when the mRNA of interest is present in the patient specimen. Generally, the oligonucleotide probes are immobilized on a substrate. The substrate-bound oligonucleotide probes thereby “capture” or immobilize complementary mRNA. The patient specimen remains in contact with the substrate-bound oligonucleotide probes for a period of time sufficient to ensure that hybridization to the oligonucleotide probes is complete. One skilled in the art can determine necessary “incubation” times, but a time of from about 0.25 hours to about 3.0 hours is preferred.

After a sufficient incubation time has elapsed, the patient specimen is washed with a suitable washing solution so as to remove unhybridized material. Washing techniques are well-known and/or can be readily determined by one of ordinary skill in the art. Typically, a washing fluid is employed that comprises a buffer solution, and, inter alia, a detergent. The buffer solution may be any conventional solution known in the art suitable for removing unhybridized material. Preferred buffer solutions contain one or more salts of alkali metals. Particularly preferred buffer solutions contain sodium chloride, sodium citrate or combinations thereof. The detergent may be any detergent that is suitable for washing unbound oligonucleotide probes. Exemplary detergents are non-ionic polyoxyethylene-based detergents, e.g., Brij® and Triton®. Similar non-ionic detergents also suitable for use in the present invention are sold under the trade names of Tween®, Genapol®, Igepal Ca®, Thesit®, and Lubrol® (all available from commercial suppliers such as Sigma Corp., St. Louis, Mo.).

Washing is generally carried out at least one, preferably two, and most preferably three times. Preferred temperatures for carrying out the wash step range from about 21° C. to about 60° C. Optimally, the wash step is carried out at room temperature.

Northern blot assays can also be used to detect and measure mRNA markers. Generally, mRNA molecules are separated on the basis of size and charge by, for example, gel electrophoresis. The mRNA molecules are then immobilized onto a suitable substrate such as nitrocellulose by contacting the gel with the substrate and allowing capillary action to transfer the mRNA from the gel to the substrate. Label probes, similar to those discussed above for sandwich-based assays, are added to the substrate and allowed to anneal to complementary mRNA. Unbound label probes are then washed away from the substrate. Detection of the mRNA labeled complexes can be accomplished as described above for sandwich-based assays.

mRNA may also be measured using homogenous or solution-phase assays. Solution-phase assays are performed without a solid substrate and often use FRET dye pairs. As is known in the art, the dye pairs emit a specific frequency of light when the dye pair is in proximity to each other (generally about 0.5 nm to about 10 nm) due to the emission of the first dye that is absorbed by the second dye, which, in turns, emits a second frequency. If the dye pair is not in proximity to each other, a different frequency is detected. Thus, it is possible to detect and measure mRNA by designing the assay and probes such that, for example, the dye pair is proximal to each other when the mRNA is present and not proximal when the dyes are not. Common FRET pairs include, but are not limited to, pairs formed from (1) 4,4-difluoro-5,7-dimethyl-4-bora-3a,4a-diaza-s-indacene-3-propionic acid and/or 4,4-difluoro-5,7-dimethyl-4-bora-3a,4a-diaza-s-indacene-3-pentanoic acid (e.g., as may be obtained from Molecular Probes, Inc. under the BODIPY FL® and BODIPY FL C5® tradenames, respectively), or salts or esters thereof, (2) fluorescein and tetramethylrhodamine, (3) fluorescein and N-(iodoacetyl)-N′-(5-sulfo-1-naphthyl)ethylenediamine (IAEDANS), (4) [(2′aminoethyl)-amino]naphthalenesulfonic acid (EDANS) and 4-[[4′-(dimethylamino]phenyl]azo]-benzoic acid) (DABCYL), and the like. A typical assay of this type includes a “molecular beacon” assay. This assay and other assays are described in, for example, Heller et al. EP 0070685, Morrison et al. (1993) Biochemistry 32(12):3095-3104, U.S. Pat. No. 4,776,062 to Diamond et al., U.S. Pat. No. 5,210,015 to Gelfand et al., U.S. Pat. No. 5,538,848 to Livak et al., and U.S. Pat. No. 5,925,517 to Tyagi et al.

In addition, mRNA may be measured by RT-PCR methods. RT-PCR encompasses making cDNA based on the mRNA present in the sample followed by measurement. Such techniques are well known in the art. Briefly, an excess of the four deoxynucleotide triphosphate molecules (i.e., deoxyadenosine triphosphate, deoxycytidine triphosphate, deoxythymidine triphosphate and deoxyguanosine triphosphate), and primer, i.e., an oligo-dT primer, are added to the patient specimen. After separation from the mRNA strand (by denaturing in a basic medium, for example), the resulting oligonucleotide is a single-stranded DNA complementary to the original mRNA sequence. Thereafter, a DNA polymerase may be added in the presence of an excess of the four deoxynucleotide triphosphate molecules and a primer to create double-stranded DNA. For more specific procedures on RT-PCR and preparing cDNA see, for example, Gerard et al. (1997) Mol. Biotechnol. 8(1):61-77 and Ando et al. (1997) J. Clin Microbiol. 35(3):570-577.

Thereafter, the double-stranded DNA can be denatured into single-stranded DNA wherein one of the two strands is essentially a DNA corresponding to the original mRNA. The DNA, however, is less susceptible to degradation and may therefore be used as a more stable surrogate for mRNA in determining the amount of the mRNA in a patient specimen. A variety of methods to detect DNA are known and may be used to detect and determine the amount of the corresponding mRNA originally contained in the patient specimen. As will be appreciated, many of the methods for detecting and measuring mRNA described herein can be adapted to detect and measure DNA.

TMA assays are similar to RT-PCR assays in that reverse transcriptase is added to the prepared patient specimen to create cDNA of the target mRNA. In TMA, however, a RNA polymerase is added to synthesize RNA amplicons using cDNA as a template. Each of the newly synthesized amplicons reenters the TMA process and serves as a template for a new round of replication. Thus, the TMA process results in the effective amplification of the mRNA. The RNA amplicons are then detected and measured by labeled probes complementary for the RNA amplicons. Similar TMA-based assays are described in the literature. See, for example, Pasternack et al. (1997) J. Clin. Microbiol. 35(3):676-678.

mRNA may also be measured using a technique known as NASBA®, which is a homogenous amplification process. Briefly, three enzymes—reverse transcriptase, RNase H, and T7 RNA polymerase—and two primers are added in a single reaction vessel containing mRNA from the sample. The first primer contains a 3′ terminal sequence that is complementary to a sequence on the mRNA and a 5′ terminal sequence that is recognized by the T7 RNA polymerase. In combination, these reagents result in the synthesis of multiple copies of mRNA that can then be measured by adding an appropriate labeled probe. Thus, those skilled in the art can use NASBA® to measure the mRNA markers. This type of assay is well-known in the art and is described in, for example, Davey et al. EP 0329822.

In RNAs protection assays, labeled oligonucleotide probe is added to the prepared patient specimen resulting in the hybridization between the labeled probe and any complementary mRNA. The sample is then treated with RNase to degrade all remaining single-stranded mRNA. Hybridized portions of the probe will be protected from digestion. Unhybridized fragments can be separated from the larger, hybridized complexes that bear a label by, for example, electrophoresis. The label can then be measured. If the probe is added at a molar excess, e.g., at least twice molar excess, with respect to the mRNA, the resulting signal is proportional to the amount of mRNA in the sample.

The assays and techniques described above require a variety of olgionucleotide probes. Sequences of the oligonucloetide probes are determined using techniques known in the art. The oligonucleotide probe sequence will be determined based on the known sequence of the mRNA of interest. Actual sequences of mRNAs can be determined experimentally or obtained by accessing an appropriate database such as the GenBank® database (National Center for Biotechnology Information, Bethesda Md.). Those regions of the sequences intended to be involved with binding (and thus are complementary to another sequence of oligonucleotides) will each be at least 15 nucleotides, usually at least 25 nucleotides, and not more than about 1000 nucleotides. Typically, the binding sequences will be approximately 25 nucleotides in length. They will normally be chosen to bind to different sequences of the analyte and/or to specific and different portions of the various probes.

Probes with a second binding sequence, e.g., intermediate oligonucleotide probes, are selected to be substantially complementary to the appropriate region of the probe. The second binding sequence may be contiguous to the first binding sequence or may be spaced therefrom by an intermediate noncomplementary sequence. The probes may include other noncomplementary sequences if desired. These noncomplementary sequences, however, must not hinder the binding of the binding sequences or result in nonspecific binding.

The probes may be prepared by oligonucleotide synthesis or by cloning, with the former preferred. As is now well-known in the art, methods for synthesizing oligonucleotides typically involve sequential addition of 3′-blocked and 5′-blocked nucleotide monomers to the terminal 5′-hydroxyl group of a growing oligonucleotide chain, wherein each addition is effected by nucleophilic attack of the terminal 5′-hydroxyl group of the growing chain on the 3′-position of the added monomer, which is typically a phosphorus derivative such as a phosphotriester, phosphoramidite, or the like. Such methodology will be known to those skilled in the art and is described in the pertinent texts and literature, e.g., in D. M. Matteuci et al. (1980) Tet. Lett. 521:719, U.S. Pat. No. 4,500,707 to Caruthers et al., and U.S. Pat. Nos. 5,436,327 and 5,700,637 to Southern et al.

Protein Markers:

As previously indicated, certain proteins may be used as markers. Techniques for obtaining patient specimens are the same as provided above with respect to mRNA. The protein marker may be present intracellularly and/or extacellularly in the patient specimen. For extracellular protein markers, the present method may be carried out using the patient specimen without lysing cells. For intracellular markers, the present method is preferably carried out with samples containing white blood cells including monocytes, dendritic cells, lymphocytes, polymorphonuclear leukocytes and combinations thereof that are lysed. Lysing of cells, without degrading proteins in the sample, may take place using techniques well-known to those skilled in the art and include exposing the sample to hyptonic conditions. Once the patient specimen is prepared, the proteins are measured using any art-known method such as, for example, immunoassay, centrifugation, electrophoresis, enzyme immunoassay, high performance liquid chromatography (HPLC), size exclusion chromatography, solid-phase affinity and Western blotting. As with the above methodology pertaining to detection of mRNA markers, flow cytometry methods may be used to expediently detect a protein marker or other marker in a specimen.

Preferably, a protein marker is measured using an immunoassay. Any art-known immunoassay that can detect proteins may be used. Immunoassays involve techniques that make use of the specific binding between an epitope on a molecule and its homologous antibody in order to identify and preferably quantify a substance in a sample. Thus, the immunoassays used to measure protein markers make use of specific binding between the protein marker and a corresponding antibody directed against the protein marker. One method for detecting protein markers involves placing the patient specimen on a slide, adding an appropriately labeled antibody, washing unbound labeled antibody, and viewing the specimen with an appropriate device, e.g., microscope, for the presence of bound protein.

Another approach involves substrate-bound antibodies directed against a particular protein marker are contacted with the patient specimen in order to immobilize the particular protein marker. After unbound protein is washed, a second labeled antibody directed to a different epitope on the protein marker is contacted with the immobilized protein. The labeled antibody is detected and quantified. Specific immunoassays are well known to those of ordinary skill in the art. For example, enzyme immunoassays such as an enzyme-linked immunosorbant assay (ELISA) employ an enzyme as the detectable label.

Antibodies specific for the protein may be available commercially or produced using art-known methods such as monoclonal or polyclonal production of antibodies. By way of a nonlimiting example, a protein is injected into a host, e.g., rabbit or mouse, and its spleen is removed several weeks later. In the presence of ethylene glycol, spleen cells from the host are added to myeloma cells that lack hypoxanthine-guanosine phosphotibosyl transferase (HGPRT). In a medium that contains hypoxanthine, aminopterin and thymine (“HAT medium”), only fused cells survive because the unfused spleen cells do not grow in vitro and unfused myeloma cells cannot create new nucleotides in the HAT medium without HGPRT. The fused cells can then be tested for the production of the desired antibody and subsequently separated and cultured. The result is a supply of antibodies directed against the protein.

In addition, phage display of antibodies may be used. In such a method, single-chain Fv (scFv) or Fab fragments are expressed on the surface of a suitable bacteriophage, e.g., M13. Briefly, spleens cells of a suitable host, e.g., mouse, that has been immunized with a protein are removed. The coding regions of the VL and VH chains are obtained from those cells that are producing the desired antibody against the protein. These coding region are then fused to a terminus of a phage sequence. Once the phage is inserted into a suitable carrier, e.g., bacteria, the phage displays the antibody fragment. Phage display of antibodies may also be provided by combinatorial methods known to those skilled in the art. Antibody fragments displayed by a phage may then be used as part of an immunoassay.

Measuring protein markers (with or without immunoassay-based methods) may also include separation of the proteins: centrifugation based on the protein's molecular weight; electrophoresis based on mass and charge; HPLC based on hydrophobicity; size exclusion chromatography based on size; and solid-phase affinity based on the protein's affinity for the particular solid-phase that is use. Once separated, the proteins may be identified based on the known “separation profile,” e.g., retention time, for that protein and measured using standard techniques. Alternatively, the separated proteins may be detected and measured by, for example, a mass spectrometer.

One type of assay that uses both separation and immunoassay techniques is the Western blot. In a Western blot, proteins located on a gel following electrophoretic separation are transferred by blotting onto a suitable substrate, e.g., nitrocellulose. A substrate-labeled antibody specific for the protein marker of interest is added to the sheet. Thereafter, rinsing the substrate with a second labeled antibody specific for the first antibody produces a detectable complex. As will be appreciated, variations of the assay are possible using different labels, substrates, etc.

Depending on the assay design, the antibodies may be labeled with the same or similar moieties described above with respect to mRNA. Furthermore, the techniques described for coupling a label to an antibody are well-known in the art and are discussed, infra.

Detection and Measurement of Markers:

Because quantitation of each marker is desired, any art-known method of quantifying the markers may be used. For example, a mass spectrometer may be used. In addition, a labeled biomolecular probe, e.g., an oligonucleotide probe (to detect an mRNA marker) or an antibody (e.g., used in an immunoassay for detecting a protein marker), may be used to measure a marker. Depending on the assay format, a plurality of identical biomolecular probes may be used to detect a given marker. Generally, the amount of each type of a labeled biomolecular probe present must be sufficient to bind to substantially all of a given marker in the sample. Such a quantity can be determined experimentally by one skilled in the art, but it is preferred that about 1 pmoles to about 1000 pmoles are used, more preferably about 10 pmoles to about 500 pmoles. In this way, substantially all of a given marker in the sample forms a probe-marker complex with the complementary biomolecular probe.

Once binding is complete, the amount of each marker present is determined by measuring the quantity of each different probe-marker complex. Measuring the quantity of a probe-marker complex may be carried out using any art-known method. In some assay formats, a second label biomolecular probe is added to the sample under binding conditions. The label biomolecular probe binds to 1) a region on the probe-marker complex or 2) a portion of an intermediary biomolecular probe that is directly or indirectly coupled the probe-marker complex. Thus, if the probe is labeled, the complex can be detected directly. When the probe is not labeled, additional layers of probe can provide for indirect detection of the marker. As will be appreciated by those skilled in the art, intermediary biomolecular probes, particularly oligonucleotide probes, may serve as a means for amplifying a signal by forming branches. The branched structure provides multiple binding sites for other label probes, thus increasing the strength of the signal by increasing the ratio of label to marker. This approach is commonly referred to as branched-oligonucleotide hybridization. See, for example, Urdea et al. (2000) Branched-DNA (bDNA) Technology in Kessler C., ed., Nonradioactive Analysis of Biomolecules, New York, Springer-Verlag:388-395.

Labeling, e.g., through probes, provides a detectable and measurable signal, thereby allowing for the quantitation of a marker present in the sample. Different labels may be used to allow for differentiation of signals if the measurement step is to be carried out simultaneously among several markers. The label may provide a direct signal, such as emission of radiation by a radioactive isotope (e.g., ³²P). Alternatively, the label may provide an indirect signal, such as production of a reaction product by an enzyme that catalyzes a reaction upon addition of the corresponding substrate. The labels may be bound, covalently or non-covalently, to the label biomolecular probe. For oligonucleotides, the label may be bound as individual members of the complementary sequence or may be present as a terminal member or terminal tail having a plurality of labels. For antibodies, the label may be coupled to the Fc unit of the antibody using techniques well-known in the art. Various means for providing labels bound to a biomolecular probe have been reported in the literature. See, for example, Leary et al. (1983) Proc. Natl. Acad. Sci. USA 80:4045; Renz et al. (1984) Nucl. Acids. Res. 12:3435; Richardson et al. (1983) Nucl. Acids. Res. 11:6167; Smith et al. Nucl. Acids. Res. (1985) 13:2399; Meinkoth et al. (1984) Anal. Biochem. 138:267.

Labels that may be employed include fluorescers, chemiluminescers, dyes, enzymes, enzyme substrates, enzyme cofactors, enzyme inhibitors, enzyme subunits, metal ions, radioactive moieties and the like. Illustrative specific labels include BODIPY®, biotin, cascade blue, coumarin, cyanine dyes (e.g., Cy3™, Cy5™, etc.), dioxetane, eosin, fluorescein, rhodamine, Texas red, phycoerythrin, umbelliferone, luminol, NADPH, NBD, Oregon Green, α,β-galactosidase, horseradish peroxidase, and alkaline phosphatase, among others. Preferably the label is a chemiluminescer or a fluorescer, e.g., fluorescein. Once the label probes or labeled mRNAs hybridize to their complementary sequences, unbound label probes and/or unbound labeled mRNAs are generally removed. Removal is effected by washing and may be carried out as described above.

Detection of the label can be accomplished by any art-known means and is dependent upon the nature of the label. For fluorescers, a number of fluorometers are commercially available. For chemiluminescers, luminometers or films are used. With enzymes, a fluorescent, chemiluminescent, or colored product can be determined fluorometrically, luminometrically, spectrophotometrically or visually (if visually, preferably with the aid of a microscope such as a confocal microscope). For radioactive moieties, films and emission detectors can be used. For the present method, it is preferred that a luminometer, confocal microscope or fluorometer is used to detect an appropriate label.

The detected signal correlates with the amount of marker in the patient specimen. Even for those assays in which an mRNA marker is amplified, e.g., RT-PCR, TMA and NASBA®, the relative amount of each mRNA copy in the sample remains substantially constant. Preservation of the relative amounts of each mRNA is possible since all mRNAs present are amplified relative to the amounts of each mRNA initially contained in a sample. As will be appreciated by those skilled in the art, direct measurement of mRNA may be difficult when low levels of the mRNA of interest is in the patient specimen. Amplification of the mRNA allows for facile detection and quantification.

Those having ordinary skill in the art can determine the quantity of a marker present in a sample based on detected signals. For example, measuring the signals from a range of controlled amounts of marker allows for the interpolation or extrapolation of the signal detected from a sample containing an unknown amount of marker. It should be noted that the determination of the absolute amount of marker in the sample is not necessary, and that the ability to measure relative amounts of marker is sufficient.

Determination of the Infection:

Once each of a plurality of markers of interest is quantified, a marker profile is identified based on the quantity of each marker. The marker profile may be limited to simply the measured amount of each marker. Such a profile is quantitative in nature. Alternatively, the marker profile may be qualitative in nature, based on a comparison of the measured amount of each marker to a previously established normal range. The normal range of any given marker in healthy individuals is generally established prior to carrying out the present method. Establishing the normal range for a particular marker can be readily accomplished by one of ordinary skill in the art. For example, the techniques described above in Section C can be used to measure the marker of interest in healthy individuals in order to establish a normal range or baseline amount for that marker.

The normal range may be provided as a range based on statistical analysis (e.g., finding the standard deviation) of the values obtained from healthy individuals. Thus, any value that falls within the normal range is considered normal while values outside the range are considered abnormal.

When the quantity of marker obtained from an individual suspected to be suffering from an infectious pathogen is outside the range established for healthy individuals, that amount is identified as abnormal. Preferably, the abnormal amount represents a greater than two-fold difference, more preferably greater than four-fold difference, and most preferably greater than ten-fold difference than a normal amount.

If the marker profile is indicative of an infection, the profile is then used to determine the type of infectious pathogen. This step is preferably accomplished by comparing the marker profile as a whole to previously established profiles corresponding to known types of infections. If the marker profile does not correspond to any previously established profile then a determination is made that the patient is not infected with any of those infections for which the corresponding profiles are known.

Advantageously, the entire method of the present invention is expedient, particularly in comparison to prior diagnostic techniques for determining types of infectious pathogens. Once all standards and reagents are prepared, the method typically takes from about 5 minutes to 12 hours, more preferably from about 15 minutes to 3 hours, and most preferably from 30 minutes to 1.5 hours, from obtaining the body fluid samples to final determination of the infectious pathogen.

Identification of Markers:

The markers of interest are those that correspond to signals of the innate immune response associated with specific types of infectious pathogens. Any method that can detect qualitative and/or quantitative differences in the amount of markers produced from a cell taken from an infected individual may be used. Such methods are well-known to those skilled in the art.

One method includes comparing (a) the expression of genes in a specimen obtained from a patient infected with the infectious pathogen to (b) the expression of genes in a specimen obtained from an individual who is not infected. By comparing the two, i.e., determining which genes are expressed in the sample taken from an infected patient to those in an uninfected individual, it is possible to identify those genes of the innate immune system that become expressed upon exposure to a particular infectious pathogen. From such information, it is possible to determine those mRNAs that are suitable to be used as markers for a particular type of infectious pathogen. Furthermore, the corresponding protein marker can then be determined based on the mRNA sequence.

In another method, a protein marker can be identified by comparing (a) the proteins present in a specimen obtained from patient who is infected with the infectious pathogen to (b) the proteins present in a specimen obtained from an individual who is not infected with the infectious pathogen. Any protein present in (a) and not in (b) indicates a protein associated with the presence of an infectious pathogen. Once this protein is known, it may be used as a protein marker. The proteins may be intracellular proteins, extracellular proteins or both. Comparison of the proteins from infected and healthy individuals may be accomplished through any art-known method. For example, commercial protein chips are available. In addition, comparison of the gels from gel electrophoresis can be used to identify a protein present in a sample from an infected individual and not in a healthy individual.

As will be appreciated, samples can be taken from individuals suffering from nearly any type of infectious pathogen and compared to healthy (control) individuals. In this way, a multitude of different markers, each specific for a particular type of pathogen, can be determined.

Utility:

The present invention is useful for determining the type of infectious pathogen causing sickness in a patient. Knowledge of the type of pathogen causing an infection allows clinicians and health care professionals to provide more specific and directed treatment. Moreover, treatment is economical as less useful or ineffective therapies are avoided.

Furthermore, the invention is useful in providing timely information concerning an infection. Timely information concerning the nature of an infectious pathogen is critical for those patients suffering from very aggressive infections or infections that are difficult to diagnose. Thus, the invention is useful in point-of-care settings in which clinicians need to provide specific and timely treatment.

For example, patients presenting with suspected nosocomial (i.e., community-acquired pneumonia), meningitis, sepsis and wound infections are usually treated with broad-spectrum antibiotics. Using conventional diagnostic approaches, the pathogen may never be identified. The present invention solves this problem by identifying the type, e.g., gram-positive or gram-negative bacteria, causing the infection. Broad-spectrum antibiotics may not be required if, for example, it is determined that the infection is caused by gram-positive bacteria. In this case, therapeutic agents such as erythromycin or vancomycin that are generally reserved for gram-positive bacteria may be administered to the patient rather than a broad-spectrum antibiotic.

Preferably, the method of the present invention is carried out using a specifically designed assay kit. The assay kit includes a plurality of biomolecular probes, a plurality of label probes and written instructions for carrying out the assay. The biomolecular probes are each complementary to a first region of different markers and consequently forms probe marker complexes under suitable binding conditions. The label probes each have a region that binds to either a region a probe-marker complex or a region of an intermediary biomolecular probe that is directly or indirectly coupled to a probe-marker complex. The assay kit may have a format as discussed herein or may have any other format suitable for assisting in the detection and measurement of a marker. The biomolecular probes may or may not be attached to a substrate.

The assay kit preferably employs a multitude of different probes, each designed to identify a series of different markers. Such “multiplex” assays have the advantage of quickly screening for a variety of infectious pathogens with a single blood sample from a patient. Thus, it is preferred that the assay detect and measure from 1 to about 500, more preferably about 10 to about 100, and most preferably about 50 to about 100 different markers.

The kits may also include any necessary reagents. These reagents will typically be in separate containers in the kit. The kit may include a denaturation reagent for denaturing the analyte, hybridization or binding buffers, wash solutions, enzyme substrates, and negative and positive controls.

It is to be understood that while the invention has been described in conjunction with the preferred specific embodiments thereof, the foregoing description, as well as the examples that follow, are intended to illustrate and not limit the scope of the invention. Other aspects, advantages and modifications will be apparent to those skilled in the art to which the invention pertains. All patents, patent applications, journal articles and other references cited herein are incorporated by reference in their entireties.

In the following examples, efforts have been made to ensure accuracy with respect to numbers used (e.g., amounts, temperature, etc.) but some experimental error and deviation should be accounted for. Unless indicated otherwise, temperature is in degrees C and pressure is at or near atmospheric. All components were obtained commercially unless otherwise indicated.

### EXAMPLES

The practice of the present invention will employ, unless otherwise indicated, conventional techniques of oligonucleotide hybridization, organic chemistry, and the like, which are within the skill of the art. Such techniques are explained fully in the literature.

The following examples are put forth so as to provide those of ordinary skill in the art with a complete disclosure and description of how to make and use the invention, and are not intended to limit the scope of what the inventors regard as their invention.

### Example 1

Blood samples from a series of patients were obtained and the Mx level in each sample was determined using conventional techniques. Cells from each sample were lysed, either immediately or immediately after freezing, as shown in Table 1. Bound Mx protein was detected using conventional techniques, i.e., using a capture antibody to immobilize bound Mx protein and detecting the labeled monoclonal antibody once unbound species have been washed away. Quantitation of the MxA protein was determined using conventional techniques, i.e., using calibration curves based on known amounts of Mx protein. The results are presented in Table 1.

TABLE 1

| | | | Mx level | |
|---|---|---|---|---|
| | Mx level | | (ng/ml) | |
| | (ng/ml) | | NOT | |
| PATIENT | LYSED PRIOR | | LYSED PRIOR | |
| ID | TO FREEZING | | TO FREEZING | Etiology |
| F1 | 29.72 | | 14.96 | Unknown |
| P1 | 0 | | 49.46 | Unknown |
| W1 | 82.14 | | 86.7 | Viral (Herpes Simplex |
| | | | | Virus Type 1) |
| XCA0059 | 0 | | 10.78 | Unknown |
| XCA0060 | 96.04 | | 49.3 | Viral (Adenovirus) |
| XEB0040 | 12.944.46 | | 12.94 | Unknown |
| | 1st run | 2nd run | | Unknown |
| B1 | 31.12 | 90.38 | | Unknown |
| F2 | 45.5 | 63.8 | | Unknown |
| H1 | 179.26 | 201.68 | | Unknown |
| XCA0061 | 55.68 | 85.48 | | Unknown |
| XEB0049 | 62.94 | 17.36 | | Unknown |
| XEB0052 | 72.68 | 85.86 | | Unknown |

| | | |
|---|---|---|
| XCA0021 | 728.66 | Viral (Adenovirus) |
| XCA0025 | 0 | Unknown |
| XCA0029 | 21.78 | Unknown |
| XEB0001 | 0 | Bacterial (Mycoplasma |
| | | pneumoniae) |
| XEB0002 | 0 | Bacterial (Group A. |
| | | Streptococcal Disease) |
| XEB0013 | 0 | Unknown |
| XEB0016 | 8.98 | Bacterial |
| | | (Cryptosporidium) |
| XEB0035 | 32.54 | Bacterial (Mycoplasma |
| | | pneumoniae) |
| XEB0037 | 0 | Bacterial (Bartonella) |
| XEB0048 | 75.64 | Viral (Influenza A |
| | | virus) |
| XEB0053 | 0 | Bacterial (Salmonella |
| | | typhi) |

As seen in Table 1, relatively significant Mx values were not limited to individuals suffering from viral infections. For example, the sample obtained from the patient identified as XEB0035, suffering from the bacterial infection Mycoplasma pneumoniae, had an Mx value of 32.54 ng/ml, while patient XCA0060, suffering from an adenoviral infection, exhibited an Mx value of 49.3 ng/ml. Consequently, Mx cannot serve as a single biomarker to effectively determine the type of an infectious pathogen. Instead, it is expected that a plurality of biomarkers, e.g., Mx protein in addition to one or more biomarkers, must be used in order to effectively determine infection type.

### Example 2

### Determination of a Profile Indicative of an Infection Using mRNA Markers

Blood samples (2.5 ml) are obtained from a healthy (control) individual, an individual suffering from a viral infection, and an individual suffering from a gram-positive bacterial infection. It is established that both infections began 3 hours prior to obtaining the blood sample as a consequence of exposure to the infectious pathogen.

Each sample is prepared for analysis. Human placental RNase inhibitor is added to the samples followed by centrifugation. All material other than RNA is removed from the sample. The assay is conducted using a commercially available gene chip such as the Affymetrix Hu6800 oligonucleotide array, according to the manufacturer's instructions. cDNA synthesis is carried out by converting mRNA into double-stranded cDNA using a commercially available cDNA synthesis kit (e.g., as may be obtained from Life Technologies, Carlsbad, Calif.) having all necessary reagents, e.g., nucleotides, enzymes, etc., in combination with an oligo(dT) primer incorporating an RNA polymerase promoter site. Labeled RNAs are made from the cDNA library in an in vitro transcription reaction by incorporating fluorescein-labeled rUTP (along with unlabeled nucleotides). Unincorporated nucleotides are removed by chromatography (Sephadex S200, available from Amersham Pharmacia Biotech, Inc., Piscataway N.J.). Each sample, now containing labeled RNA, is heated (to approximately 40° C.) in a hybridizing solution (100 mM MES [2-(N-morpholino)-ethanesulfonic acid], 1 M NaCl, 20 mM EDTA [ethylenediaminetetraacetic acid], and 0.01 wt. % TWEEN® 20) and placed in contact with a separate gene chip. Once hybridization is complete, each chip is washed and read, e.g., using a confocal laser microscope (available from Affymetrix, Santa Clara, Calif.).

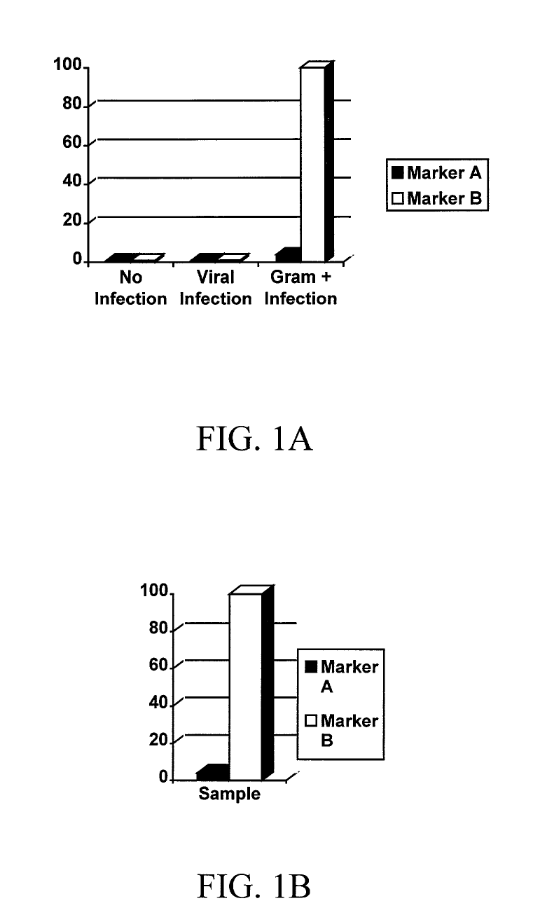

The results of the assay demonstrate that for the individual with no infection and the individual suffering from a viral infection, negligible amounts (less than 1 pM) of a first mRNA (Marker A) and a second mRNA (Marker B) were detected. However, in the sample taken from the individual infected with gram-positive bacteria, 4 pM of the first mRNA (Marker A) and 100 pM of the second mRNA (Marker B) are measured. See FIG. 1A.

Thus, normal levels for these two mRNAs are determined to be less than 1 pM. Furthermore, it is determined that a gram-positive bacterial infection is identified by a profile having approximately 4 pM of Marker A and approximately 100 pM of Marker B.

### Example 3

### Identifying the Type of Infectious Pathogen in an Patient Suspected of Suffering from an Infection

A blood sample is taken from a patient who is suspected to be suffering from an infectious pathogen. The sample is prepared and analyzed according to procedures set forth in Example 2. The results are obtained in less than 3 hours.

The results of the assay indicate that the sample obtained from the patient has a marker profile of 4 pM of Marker A and 100 pM of Marker B. See FIG. 1B. Based on the profile identified for gram-positive bacterial infections established in Example 2, it is concluded that the patient is suffering from an infection of gram-positive bacteria. An antibiotic specific for gram-positive infections is administered to the patient. Two weeks later, culture analysis reveals that the infection is Listeria monocytogenes, a gram-positive bacterium.

### Example 4

### Multiplex Assays

Using the procedures of Example 2, additional profiles indicative of infections based on mRNA markers are determined for other infectious pathogens, e.g., viral, fungal, etc. Once a number of profiles are determined, oligonucleotide probes for each mRNA marker are coupled to a solid substrate such as a chip or plurality of different beads. When beads are used, each bead is differently colored for ease of analysis. Thereafter, a single blood sample taken from a patient suspected to be suffering from an infection is assayed. In this way, a spectrum of mRNAs are measured to identify several marker profiles that are used to determine the type infectious pathogen causing illness in a patient. Once the type of infectious pathogen is determined, the clinician initiates appropriate therapeutic intervention.

We claim:

1. A method for determining the type of an infectious pathogen in a patient who is suspected to be suffering from an infectious pathogen, comprising:
a) measuring the amounts of each of a plurality of markers in a specimen obtained from the patient, wherein each of the markers is produced by the patient as a part of that patient's innate immune response to the presence of the infectious pathogen and the plurality of markers is indicative of the type of the infectious pathogen;
b) identifying a marker profile based on the measured amounts of each of the plurality of markers; and
c) if the marker profile is indicative of an infection, then determining the type of infectious pathogen from the marker profile.

2. The method of claim 1, wherein at least one of the plurality of markers is an mRNA.

3. The method of claim 2, wherein each marker is an mRNA.

4. The method of claim 2, wherein the measuring step is performed using techniques selected from the group consisting of sandwich hybridization, branched-oligonucleotide hybridization, Northern blotting, solution phase assay, reverse transcriptase-polymerase chain reaction, transcription-mediated amplification, nucleic acid sequence-based amplification and RNAse protection assay.

5. The method of claim 4, wherein the technique is selected from the group consisting of sandwich hybridization, reverse transcriptase-polymerase chain reaction, transcription-mediated amplification.

6. The method of claim 1, wherein at least one of the plurality of markers is a protein.

7. The method of claim 6, wherein each marker is a proteins.

8. The method of claim 6, wherein the measuring step is performed using techniques selected from the group consisting of immunoassay, centrifugation, electrophoresis, enzyme immunoassay, high performance liquid chromatography (HPLC), size exclusion chromatography, solid-phase affinity and Western blotting.

9. The method of claim 8, wherein the technique is selected from the group consisting of immunoassay, electrophoresis, HPLC and Western blotting.

10. The method of claim 9, wherein the technique is an immunoassay technique.

11. The method of claim 1,wherein the plurality of markers includes at least one mRNA and at least one protein.

12. The method of claim 1, wherein the measuring step is performed using a label probe that is specific for a single marker.

13. The method of claim 12, wherein the label probe is either a labeled oligonucleotide or a labeled antibody.

14. The method of claim 13, wherein the label probe includes a detectable label selected from the group consisting of fluorescers, chemiluminescers, dyes, enzymes, enzyme substrates, enzyme cofactors, enzyme inhibitors, enzyme subunits, metal ions, and radioactive isotopes.

15. The method of claim 1, wherein the specimen obtained from the patient comprises a body fluid.

16. The method of claim 15, wherein the body fluid is selected from the group consisting of blood, sputum, urine and fractions of whole blood.

17. The method of claim 15, wherein the body fluid contains cells.

18. The method of claim 17, wherein the cells comprise white blood cells.

19. The method of claim 18, wherein the white blood cells are selected from the group consisting of monocytes, dendritic cells, lymphocytes, polymorphonuclear leukocytes and combinations thereof.

20. The method of claim 1, wherein the specimen obtained from the patient comprises extracellular fluid.

21. The method of claim 1, wherein the infectious pathogen is bacterial.

22. The method of claim 21, wherein the infectious pathogen is gram-positive bacteria.

23. The method of claim 21, wherein the infectious pathogen is gram-negative bacteria.

24. The method of claim 1, wherein the infectious pathogen is fungal.

25. The method of claim 1, wherein the infectious pathogen is viral.

26. The method of claim 1, wherein more than two markers are used to determine the type of infectious pathogen.

27. An assay kit for determining the presence of an infectious pathogen in a patient, comprising:
a) a plurality of biomolecular probes each complementary to a different marker within a plurality of markers, such that one or more probe-marker complexes is formed under binding conditions, is at least partially indicative of the presence of an infectious pathogen;
b) a plurality of label probes each having a region that binds directly or indirectly to one or more probe-marker complexes; and
c) written instructions for carrying out the assay.

28. The assay kit of claim 27, wherein the biomolecular probes are oligonucleotide probes and the markers are mRNAs.

29. The assay kit of claim 28, having an assay format selected from the group consisting of a sandwich hybridization assay, branched-oligonucleotide hybridization, Northern blotting, solution-phase assay, reverse transcriptase-polymerase chain reaction, transcription-mediated amplification, nucleic acid sequence-based amplification and RNAse protection assay.

30. The assay kit of claim 29, having an assay format selected from the group consisting of sandwich hybridization assay, reverse transcriptase-polymerase chain reaction, transcription-mediated amplification.

31. The assay kit of claim 27, wherein the biomolecular probes are antibody probes and the markers are proteins.

32. The assay kit of claim 31, having an assay format selected from the group consisting of immunoassay, centrifugation, electrophoresis, enzyme immunoassay, high performance liquid chromatography (HPLC), size exclusion chromatography, solid-phase affinity and Western blotting.

33. The assay kit of claim 32, having an assay format selected from the group consisting of immunoassay, electrophoresis, high performance liquid chromatography (HPLC) and Western blotting.

34. The assay kit of claim 33, having an immunoassay format.

35. The assay kit of claim 27, wherein the label probe includes a detectable label selected from the group consisting of fluorescers, chemiluminescers, dyes, enzymes, enzyme substrates, enzyme cofactors, enzyme inhibitors, enzyme subunits, metal ions and radioactive isotopes.

36. The assay kit of claim 27, wherein the plurality of markers includes at least one mRNA and at least one protein.

37. A method for identifying a marker that is indicative of the presence of an infectious pathogen in a patient, comprising:
comparing (a) the genome-wide expression of genes of a specimen obtained from a patient who is infected with the infectious pathogen to (b) the genome-wide expression of genes of a specimen obtained from an individual who is not infected with the infectious pathogen, wherein a gene expressed in (a) and not in (b) indicates a gene associated with the presence of the infectious pathogen; and
determining from the gene associated with the presence of an infectious pathogen, the corresponding marker.

38. The method of claim 37, wherein the marker is an mRNA.

39. The method of claim 37, wherein the marker is a protein.

40. The method of claim 37, wherein both specimens comprise white blood cells.

41. A method for identifying a protein marker that is indicative of the presence of an infectious pathogen in a patient, comprising:
comparing (a) the proteins present in a specimen obtained from a patient who is infected with the infectious pathogen to (b) the proteins present in a specimen obtained from an individual who is not infected with the infectious pathogen, wherein a protein present in (a) and not in (b) represents a protein marker that is indicative of the presence of an infectious pathogen.

42. The method of claim 41, wherein the comparison step comprises use of gel electrophoresis.

43. The method of claim 41, wherein both specimens comprise white blood cells.
